(19) United States
(12) Patent Application Publication
(10) Pub. No.: US 2015/0328354 A1
(43) Pub. Date: Nov. 19, 2015

(54) Scent Machine with Filter

(71) Applicants: Francisco J. Guerra, Lexington, AL (US); Joy Mangano, Ronkonkoma, NY (US)

(72) Inventors: Francisco J. Guerra, Lexington, AL (US); Joy Mangano, Ronkonkoma, NY (US)

(21) Appl. No.: 14/278,653

(22) Filed: May 15, 2014

(51) Int. Cl. A61L 9/12

(52) U.S. Cl. CPC A61L 9/122

(57) ABSTRACT

A scent machine includes a housing defining an interior chamber with a top cone in which is defined a top opening and a slot defined circumferentially in a wall of the top cone. The machine further includes a filter housed and a fan housed within the chamber. The slot receives a fragrance-infused disc.

### BRIEF DESCRIPTION OF THE DRAWINGS

The apparatus is described with reference to the accompanying drawings. In the drawings, like reference numbers indicate identical or functionally similar elements.

FIG. 1 is a sectional schematic of the scent machine; and

FIG. 1A is a plan view of the fragrance-infused disc.

### DETAILED DESCRIPTION

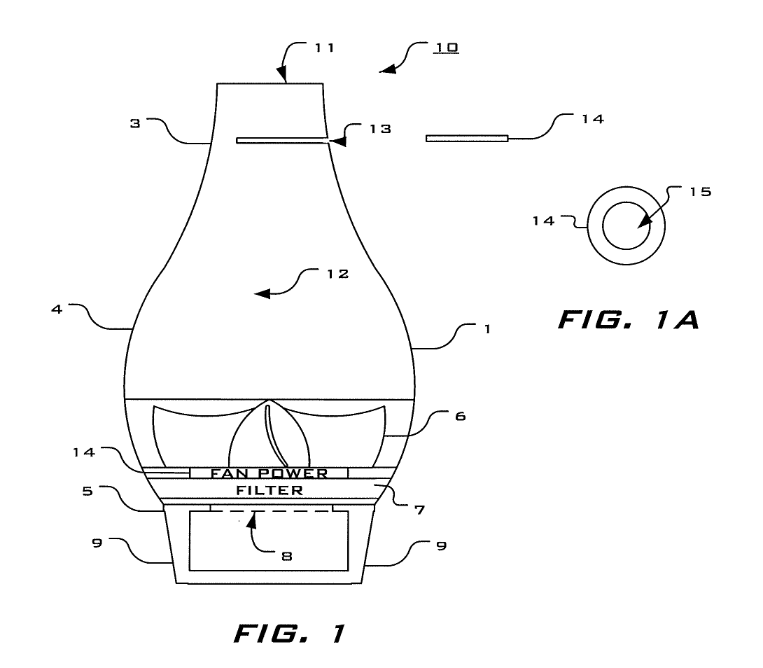

The various embodiments of the present invention and their advantages are best understood by referring to FIGS. 1 and 1A of the drawings. The elements of the drawings are not necessarily to scale, emphasis instead being placed upon clearly illustrating the principles of the invention. Throughout the drawings, like numerals are used for like and corresponding parts of the various drawings.

Furthermore, reference in the specification to “an embodiment,” “one embodiment,” “various embodiments,” or any variant thereof means that a particular feature or aspect of the invention described in conjunction with the particular embodiment is included in at least one embodiment of the present invention. Thus, the appearance of the phrases “in one embodiment,” “in another embodiment,” or variations thereof in various places throughout the specification are not necessarily all referring to its respective embodiment.

This invention may be provided in other specific forms and embodiments without departing from the essential characteristics as described herein. The embodiments described above are to be considered in all aspects as illustrative only and not restrictive in any manner.

A scent machine 10 comprises a housing 1 having a base 5 supporting a body 4 to which is coupled a top cone 3. Body 4 and top cone 3 define an open interior chamber 12. The base 5 is configured with a baffled opening 8 that allows the intake of ambient air into the housing 1. Base 5 may further comprise a plurality of elongated risers 9 to elevate the machine 10 above the surface upon which it sets in order to allow air to flow freely to the opening 8 and into the chamber 12. Risers 9 may be connected together.

The body houses a fan 6, preferably powered by a battery 16, which may be a rechargeable battery, and a filter 7. Fan 6 may also be adapted to include a power supply configured to relay power from standard residential electrical systems as would be appreciated by those skilled in the arts. The top cone 3 comprises a generally circular base section extending upward from the body 4 with an opening 11 in the top. In general operation, the fan 6 is configured to draw ambient air into the housing 1 through the baffled 8 opening in the base 5, through the filter 7, and to impel such air through the top cone 3 and out the top opening 11.

The top cone 3 is also configured with one or more slots 13 circumferentially defined in the wall of the cone 3 near the top opening 11, and downstream of the fan 6. The slot is dimensioned to receive a disc 14 comprising an annular ring with a central opening 15 and which is infused with a pleasant fragrance. Accordingly, air impelled by the fan 6 through the top cone 3 passes through the central opening 15 of the scented disc 14 that is inserted into the slot 13 and the fragrance emanating from the disc 14 becomes entraining the air stream which exits the top cone 3 through the top opening 11.

As described above and shown in the associated drawings, the present invention comprises a scent machine with air filter. While particular embodiments have been described, it will be understood, however, that any invention appertaining to the apparatus described is not limited thereto, since modifications may be made by those skilled in the art, particularly in light of the foregoing teachings. It is, therefore, contemplated by the appended claims to cover any such modifications that incorporate those features or those improvements that embody the spirit and scope of the invention.

What is claimed is:

1. A scent machine comprising:
a housing defining an interior chamber and having
a base in which is defined an air intake in communication with said chamber, and
a top cone in which is defined a top exhaust opening in communication with said chamber and a slot defined circumferentially in a wall of said top cone;
a filter housed within said chamber;
a fan housed within said chamber;
a fragrance-infused annular ring dimensioned to be received within said slot, said ring having a central opening through which air impelled by said fan may flow.

2. The scent machine of claim 1, wherein said fan is a battery-powered fan.

3. The scent machine of claim 3, wherein said fan is powered by a rechargeable battery.
